SEQUENCE LISTING (12 sequences)

SEQ ID NO: 1 - DNA, 56 bp, synthetic construct
  source 1..56: mol_type = other DNA
aattctttttgacgcataagttgtgaaatcgtgtaatttctgctatgctaggttca

SEQ ID NO: 2 - DNA, 56 bp, synthetic construct
  source 1..56: mol_type = other DNA
aattctgtcaggttttgaacttgaagaagaagtggtgtgtggctataattgagtta

SEQ ID NO: 3 - DNA, 56 bp, synthetic construct
  source 1..56: mol_type = other DNA
aattcggtcaacaattatagttgattatcgcgtaaaagtatgctacccttaagtta

SEQ ID NO: 4 - DNA, 56 bp, synthetic construct
  source 1..56: mol_type = other DNA
aattcgagaacatgtttgcgttgaaaaacttgtaaggttgggctaaaatagcgtaa

SEQ ID NO: 5 - DNA, 48 bp, synthetic construct
  source 1..48: mol_type = other DNA
aaaaaaaaggtattgtcagtgcgaaagcactgacaatacctttttttt

SEQ ID NO: 6 - DNA, 48 bp, synthetic construct
  source 1..48: mol_type = other DNA
aaaaaaaaggttgtttcagtgctttcgcactgaaacaacctttttttt

SEQ ID NO: 7 - DNA, 57 bp, synthetic construct
  source 1..57: mol_type = other DNA
ctcggtaccaaattccagaaaagagacgctttcgagcgtcttttttcgttttggtcc

SEQ ID NO: 8 - DNA, 57 bp, synthetic construct
  source 1..57: mol_type = other DNA
ctcggtaccaaagacgaacaataagacgctgaaaagcgtcttttttcgttttggtcc

SEQ ID NO: 9 - DNA, 411 bp, synthetic construct
  source 1..411: mol_type = other DNA
atgggtgaagttcagctggttgaatctggtggtggttctgttcaggcgggtggttctctgcgtctgtcttgcgcggcgtctggtgtttctaacaccgacatgatcatgatctggttccgtcaggcgccgggtaaagaacgtgaaggtgttctggcggcgatctacaaaaactctacctactacgcggactctgttaaaggtcgtttcaccatctctcaggacaacgcgaaaaacaccgtttacctgcagatgaactctctgaaaccggaagacaccgcgatctactactgcgcggcgatccgtgcggttatcggtcgtcacatccgtccgcacatctactggggtcagggtacccaggttaccgttggtggtggttctcaccaccaccaccaccacggtggttcttaatga

SEQ ID NO: 10 - DNA, 411 bp, synthetic construct
  source 1..411: mol_type = other DNA
tcattaagaaccaccgtggtggtggtggtggtgagaaccaccaccaacggtaacctgggtaccctgaccccagtagatgtgcggacggatgtgacgaccgataaccgcacggatcgccgcgcagtagtagatcgcggtgtcttccggtttcagagagttcatctgcaggtaaacggtgtttttcgcgttgtcctgagagatggtgaaacgacctttaacagagtccgcgtagtaggtagagtttttgtagatcgccgccagaacaccttcacgttctttacccggcgcctgacggaaccagatcatgatcatgtcggtgttagaaacaccagacgccgcgcaagacagacgcagagaaccacccgcctgaacagaaccaccaccagattcaaccagctgaacttcacccat

SEQ ID NO: 11 - protein, 135 aa, synthetic construct
MGEVQLVESGGGSVQAGGSLRLSCAASGVSNTDMIMIWFRQAPGKEREGVLAAIYKNSTYYADSVKGRFTISQDNAKNTVYLQMNSLKPEDTAIYYCAAIRAVIGRHIRPHIYWGQGTQVTVGGGSHHHHHHGGS

SEQ ID NO: 12 - protein, 9 aa, synthetic construct
HHHHHHGGS

(19) United States
(12) Patent Application Publication
(10) Pub. No.: US 2024/0401064 A1
(43) Pub. Date: Dec. 5, 2024

(54) BACTERIAL DELIVERY OF ANTIBODIES, ANTIBODY DERIVATIVES, AND POLYPEPTIDES TO EUKARYOTIC CELLS

(71) Applicant: SiVEC Biotechnologies, Inc., Fort Collins, CO (US)

(72) Inventors: Lyndsey M Linke, Fort Collins, CO (US); Ashley B Williams, Austin, TX (US); Darcy Mora, Fort Collins, CO (US); Madeline Cox, Davie, FL (US); Timothy Enroth, Aurora, CO (US)

(73) Assignee: SiVEC Biotechnologies, Inc., Fort Collins, CO (US)

(21) Appl. No.: 18/565,595

(22) PCT Filed: Jun. 2, 2022
(86) PCT No.: PCT/US22/31949

§ 371 Date: Nov. 30, 2023

Related U.S. Application Data
(60) Provisional application No. 63/195,982, filed on Jun. 2, 2021.

(51) Int. Cl. C12N 15/70; C07K 16/18

(52) U.S. Cl. CPC C12N 15/70; C07K 16/18; C07K 2317/569; C07K 2317/73

(57) ABSTRACT

A bacteria-mediated platform that uses invasive, non-pathogenic bacteria to both produce and intracellularly deliver antibodies, antibody derivatives, and proteins/polypeptides to targeted eukaryotic cells and tissues. The bacteria can contain a prokaryotic expression cassette encoding the protein cargo.

### CROSS-REFERENCE TO RELATED APPLICATIONS

This application is the National Stage of International Application No. PCT/US22/31949, filed Jun. 2, 2022, which application claims the benefit of U.S. Provisional Application No. 63/195,982 filed Jun. 2, 2021.

### REFERENCE TO AN ELECTRONIC SEQUENCE LISTING

The contents of the electronic sequence listing (0111-04-US1-SequenceData.xml; Size: 13,064 bytes; and Date of Creation: Mar. 8, 2024) is herein incorporated by reference in its entirety.

### FIELD OF INVENTION

This invention relates to treatment, prevention and diagnosis of disease. More specifically, this invention relates to a bacteria-mediated platform that uses invasive, non-pathogenic bacteria to both produce and intracellularly deliver polypeptides, proteins, antibodies, and antibody derivatives to targeted eukaryotic cells and tissues.

### BACKGROUND OF THE INVENTION

The use of agonistic and antagonistic antibodies and antibody derivatives is a rapidly growing area of interest in the development of novel therapeutic agents due in large part to their target specificity and low immunogenicity. Use of antibodies is enabling the development of therapeutic agents against diseases for which no druggable target previously existed. To date, the market is dominated by antibody-based drugs directed at extracellular targets (i.e., on the cell surface). Intracellular targets remain largely inaccessible. Despite the enormous therapeutic potential of modulating intracellular targets by agonistic or antagonistic antibodies, their use in this capacity is limited by the inability of antibodies to cross the target cell membrane and enter the cytoplasm.

To date, efficient in vivo delivery of proteins, antibodies, and antibody derivatives to targeted cells remains challenging. Most current antibody delivery strategies are based primarily on mechanical approaches (e.g., electroporation, hydrodynamic injection, microinjection) and viral vector delivery (e.g., lentivirus, adenovirus, adeno-associated virus). While useful in vitro, many of these methods cannot be easily clinically translated to animals or human patients. Non-viral delivery methods, such as liposomes and nanoparticles, are also used, but the size and number of antibodies or other proteins they can carry is extremely limited. Antibodies and antibody derivatives must be delivered more efficiently to achieve both extracellular, and in particular, intracellular therapeutic effects.

### SUMMARY OF THE INVENTION

The present invention provides a system for the production and/or intracellular delivery of polypeptides, proteins, antibodies, and antibody derivatives to eukaryotic cells using a non-pathogenic bacterial delivery platform. The delivered proteins could be agonistic or antagonistic to intracellular pathways by interacting with specific target molecules within the target cell. For example, the target molecule could be active when not complexed to an antibody derivative, but inactive when complexed. For example, the delivery of a single-domain antibody (nanobody) that complexes with a survivin molecule to inhibit cancer cell proliferation. Antibodies, antibody derivatives, and polypeptides can comprise: IgG, IgG fragments (Fab, Fab′, F(ab′)₂), V_{HH} moieties, single-chain variable fragment (scFv), di-scFv, single-domain antibody (sdAb), nanobody, camelid IgG antibody, llama IgG antibody, peptibodies, any other immune polypeptide, and any polypeptide comprised of amino-acid residues that form a therapeutic protein molecule. It is contemplated that the present invention can provide effective delivery of these molecules to a target eukaryotic cell.

In a first aspect, the present invention provides a bacteria-mediated platform that uses invasive, non-pathogenic bacteria to both produce and intracellularly deliver polypeptides, proteins, antibodies, and antibody derivatives (“protein cargo”) to targeted eukaryotic cells and tissues. The bacteria can contain a prokaryotic expression cassette encoding the protein cargo under the control of a prokaryotic promoter. The novel bacterial delivery platform for therapeutic antibodies, antibody derivatives, or proteins can provide tissue- and cell-specific delivery and intracellularization of the protein and/or antibodies in any eukaryotic cell in any cell cycle stage (dividing, non-dividing, quiescent). Targeting to desired eukaryotic cells can be controlled via the selection of invasion factors that interact with specific target cell surface moieties.

In a second aspect, the present invention provides a method for protein replacement (e.g., Enzyme Replacement Therapy) or modulating a specific activity and/or a pathway in a eukaryotic cell. In the case of protein replacement, the method can include the step of delivering a eukaryotic protein encoded and produced by the prokaryotic bacterial cell for replacing a particular eukaryotic protein that is non-functional (e.g., due to mutation) or deficient (e.g., due to haploinsufficiency). In the case of modulating a specific activity and/or pathway in a eukaryotic cell, the method can include the step of contacting the eukaryotic cell with a bacterium comprising an expression cassette encoding one or more cargo molecules, e.g., IgG, IgG fragments [Fab, Fab′, F(ab′)₂], V_{HH} moieties, single-chain variable fragment (scFv), di-scFv, single-domain antibody (sdAb), nanobody, camelid IgG antibody, llama IgG antibody, peptibodies, any other immune polypeptide, or any polypeptide comprised of amino-acid residues that form a therapeutic protein molecule, known collectively as antibodies, antibody derivatives, or proteins/polypeptides, under the control of one or more prokaryotic promoter wherein the said encoded cargo molecules are produced by the bacterium and wherein the bacterium is engineered to be invasive to the eukaryotic cell and wherein a region on the one or more antibodies or other antibody derivatives binds a target molecule within a target cell to modulate the activity of that target molecule in the eukaryotic cell.

In a third aspect, the present invention provides a bacteria-mediated platform for the production and intracellular delivery of therapeutic nanobodies. The production of structurally intact, functional antibodies in bacteria is challenging, especially due to their large size and disulfide bonds (which can only be formed in the periplasmic space of E. coli cells). For this (and many other reasons), there has been growing interest in single-domain antibodies (sdAb), also known as nanobodies. These proteins, which range in size from 12-15 kDa (versus 150-160 kDa for common antibodies) comprise only a single monomeric variable antibody domain. Importantly, as a relatively large macromolecule, nanobodies can be less sensitive to fine structures of target proteins, e.g., by binding to entire surfaces (rather than small pockets) of proteins to exert biochemical effects. For this reason, nanobodies represent a valuable mutation-recalcitrant therapeutic modality. Because of the inhibitory mechanism of nanobodies, nanobody-based therapies are expected to be less affected by acquired resistance, especially concurrent mutations in the target protein.

In a fourth aspect the present invention provides a system for generating and delivering proteins to eukaryotic cells. The system employs a bacterium that has been engineered to be invasive and has been engineered to have a least one expression cassette encoding a protein that is exogenous to the bacterium. The transcription of the nucleic acid encoding the exogenous protein is under the control of a prokaryotic promoter and terminator.

In an advantageous embodiment the prokaryotic promoter and terminator are synthetic. The synthetic promoter or terminator can have transcription-promoting or transcription-terminating activity in E. coli.

In further advantageous embodiments the encoded protein is an antibody or an antibody derivative. The antibody or antibody derivative can consist essentially of a single protein domain extracted from a multi-domain antibody. The antibody derivative consists essentially of a single V_{HH} antibody domain or a nanobody. The antibody or antibody derivative can can have a biologically active peptide (that has an effect on a living organism, tissue, cell, or biochemical process) grafted onto an Fc domain or other antibody domain (e.g., a peptibody) of the antibody or antibody derivative. The biologically active peptide can be a peptide with antioxidant, antimicrobial, immunomodulatory, cytomodulatory, and/or metabolism-altering properties or effects.

A structural domain of the antibody or antibody derivative can have an amino acid sequence that binds an epitope to target an intracellular protein, where the intracellular protein is a therapeutically relevant protein or is therapeutically relevant as a binding target for the antibody or antibody derivative.

The antibody or antibody derivative can form a complex with a target protein, thereby modulating a specific activity or cellular pathway in a eukaryotic cell by rendering the target protein biologically inactive due to the antibody obscuring, occupying, or otherwise interfering with a binding site or epitope important for target protein interactions with other molecules, while the uncomplexed target is biologically active protein.

In certain embodiments the antibody or antibody derivative binds an intracellular factor inside a cancer cell to modulate a specific activity or cellular pathway, including those related to cell survival, proliferation, and sensitivity to chemotherapeutic agents, such that the antibody or antibody derivative has an anti-tumorigenic effect. In an advantageous embodiment the intracellular factor that is bound by the antibody or antibody derivative inside a cancer cell can be a mutated HRAS, NRAS, or KRAS protein. In other words, the antibody or antibody derivative binds a mutated HRAS, NRAS, or KRAS protein.

In further advantageous embodiments binding by the antibody or antibody derivative modulates a specific activity or cellular pathway thereby enhancing the therapeutic efficacy of a chemotherapeutic agent or other therapy administered to or performed on a subject. The antibody or antibody derivative can be administered prior to, sequentially or after the chemotherapeutic agent or other therapy.

In certain embodiments the antibody or antibody derivative contains a region that binds an epitope on an apoptosis-regulating or apoptosis-related protein. the antibody or antibody derivative can contain a region that binds survivin (BIRC5), BCL-2, MCL-1, XIAP, BRUCE, or any other inhibitor of apoptosis (IAP)-family protein or protein that contains one or more characteristic BIR domains. The antibody or antibody derivative can contain a region that binds an epitope on a viral, bacterial, protozoan, or fungal protein, whereby binding the epitope inhibits viral, bacterial, protozoan, or fungal replication.

The bacterium according to the fourth aspect can be a nonpathogenic bacterium engineered to have at least one invasion factor to facilitate entry of the nonpathogenic bacterium into a eukaryotic cell or cause release of the nonpathogenic bacterium from a eukaryotic cell phagosome. The invasion factor can be encoded by an inv, hlyA, or hlyE gene or any fragment or chimeric or recombinant version thereof. the invasion factor can be a chimeric, recombinant invasion protein comprising the non-binding domains of an invasin protein fused to a binding domain from a heterologous protein. The binding domain from a heterologous protein can be a binding domain of GalNAc binding proteins, lectins, the group of cell adhesion molecules (CAMs), the group of sulfated glycosaminoglycan (GAG)-binding proteins, selectins, integrins, laminin, cadherins, fibronectin, collagens, thrombospondin, vitronectin, tenascin, apolipoproteins B, E, and A-V, lipoprotein lipase, hepatic lipase, Siglecs, galectins, immunoglobulins, and annexins, FimH, papG, PrsG, Afa-IE, DraA, MrpH, RodA, Mp1, hydrophobins, heat shock protein, CspA, hemagglutinin, neuraminidase, capsid protein, glycoproteins, and envelope proteins. In certain embodiments the invasion factor is engineered to be on the chromosome of the bacterium.

The expression cassette encoding a protein is engineered to be carried by a prokaryotic plasmid or on the chromosome of the bacterium. The expression cassette can be on a plasmid having a length of approximately 7,000 base pairs or less, approximately 6,000 base pairs or less, approximately 5,000 base pairs or less, approximately 4,000 base pairs or less, or approximately 3,000 base pairs or less. A reduction in plasmid size relative to a larger plasmid decreases plasmid-induced burden to the host bacterial cells, thereby increasing the bacterial growth rate.

The protein encoded by the invasive bacterium is delivered to the cytoplasm of a target eukaryotic cell. In certain embodiments the protein is functional in a eukaryotic cell and increases the level of protein in the target eukaryotic cell to supplement a clinically significant deficiency in the endogenous level of said protein, thereby conferring a therapeutic benefit to the target cell and the subject.

In a fifth aspect the present invention provides a method for modulating a specific activity and/or pathway in a eukaryotic cell comprising the step of contacting the eukaryotic cell with a bacterium comprising an expression cassette encoding one or more antibodies, single V_{HH} antibody domains, nanobodies, and/or other antibody derivatives under the control of one or more prokaryotic promoters wherein the bacterium is engineered to be invasive to the eukaryotic cell and wherein a region on the one or more antibodies, single V_{HH} antibody domains, nanobodies, and/or other antibody derivatives binds a target molecule whereby binding modulates the activity of the target molecule.

In a sixth aspect the present invention provides a composition comprising an engineered bacterium having a plasmid wherein the plasmid consists essentially of an origin of replication, an expression cassette encoding a selectable marker and one or more antibodies, single V_{HH} antibody domains, nanobodies, antibody derivatives or combinations thereof under the control of one or more prokaryotic promoters and terminators and wherein the bacterium is engineered to be invasive to the eukaryotic cell. The plasmid can encode more than one antibody, single V_{HH} antibody domain, nanobody, antibody derivatives or combinations thereof and at least two of the antibodies, single V_{HH} antibody domains, nanobodies, antibody derivatives or combinations thereof are under the control of a different promoter, thereby allowing for the differential expression of more than one antibody, single V_{HH} antibody domain, nanobody, antibody derivatives or combinations thereof. In an advantageous embodiment one of the antibodies, single V_{HH} antibody domains, nanobodies, antibody derivatives or combinations thereof is an anti-survivin nanobody.

In a seventh aspect the present invention provides a method for replacing or supplementing an endogenous eukaryotic protein in a eukaryotic target cell comprising the step of contacting the eukaryotic target cell with a bacterium comprising an expression cassette encoding and producing a eukaryotic protein in need of replacement or supplementation in the target cell wherein transcription of the protein is under the control of a prokaryotic promoter and wherein the bacterium is a nonpathogenic bacterium that is engineered to be invasive to the eukaryotic cell and wherein the exogenously delivered bacterially expressed eukaryotic protein has the same biological or biochemical activity as the endogenous eukaryotic protein and that this activity is present in the eukaryotic cell (i.e., the protein carries out its normal function).

### BRIEF DESCRIPTION OF THE DRAWINGS

For a fuller understanding of the invention, reference should be made to the following detailed description, taken in connection with the accompanying drawings, in which:

FIG. 1 is an illustration showing the pSiVEC2_survivin_nb plasmid. The cloned nanobody (“nb”) sequence also encodes a translationally fused 6×His affinity tag on the C-terminus of the nb. In pSiVEC2_survivin_nb, prokaryotic expression of the anti-survivin nb protein is controlled by a prokaryotic promoter (i.e., a promoter that is only active in bacterial cells). Therefore, the bacteria both produce (transcribe and translate) and deliver the anti-survivin nb.

FIG. 2 is an image that shows the results of a western blot for four independent clones of FEC21/pSiVEC2_survivin_nb (labeled #9, #10, #12, and #16). A robust band representing a protein of approximately 15 kDa, which was absent in the negative control lane, confirms strong bacterial expression of the anti-survivin nb.

FIG. 3 is a graph that shows that A549 epithelial cancer cells receiving the anti-survivin nb via the herein described bacterial delivery system have a sustained reduction in proliferation compared with cells that received the scramble sequence (no-nb control).

FIG. 4 is a graph that shows that A549 epithelial cancer cells receiving the anti-survivin nb via the herein described bacterial delivery system have 1) a sustained reduction in proliferation (compare “Control” to “Anti-survivin”) and 2) increased sensitivity to cisplatin as indicated by a more robust effect on proliferation in the presence of cisplatin (compare the effect of addition of cisplatin between “Control” and “Control+cisplatin (25 mM)” and “Anti-survivin” and “Anti-survivin+cisplatin”).

FIG. 5 is a collection of brightfield images that shows that addition of cisplatin to anti-survivin nb-treated A549 epithelial cancer cells have increased numbers of apoptotic cells that persists for at least 98 h after cisplatin addition. The triangles point to exemplary apoptotic cells

FIG. 6 is an illustration showing an E. coli-optimized, anti-survivin nanobody (“nb”) (the boxed asterisk(s) at the C-terminus indicate termination codons) sequence.

### DETAILED DESCRIPTION OF THE PREFERRED EMBODIMENT

The use of agonistic and antagonistic antibodies and antibody derivatives is a rapidly growing area of interest in the development of novel therapeutic agents due in large part to their target specificity and low immunogenicity. Use of such molecules is allowing the development of therapeutic agents against diseases for which no druggable target previously existed. To date, the market is dominated by antibody-based drugs directed at extracellular targets (i.e., targets on the cell surface). Intracellular targets remain largely inaccessible. Despite the enormous therapeutic potential of modulating intracellular targets by agonistic or antagonistic antibodies, their use in this capacity is limited by the inability of antibodies to cross the target cell membrane and enter the cytoplasm. Free antibodies in the circulation are non-specifically taken up by immune cells by virtue of their Fc domain, typically proteins found on cell surfaces, thereby limiting the ability of antibodies to reach intended intracellular targets due to systemic depletion. For these reasons, intracellular targets are often referred to as “undruggable” in the context of therapeutic antibodies, antibody derivatives, and proteins/polypeptides. This challenge has been partially met by the discovery and development of smaller antibody derivatives that lack the Fc region, e.g., single domain antibodies or nanobodies. Nevertheless, such antibody derivatives are still rapidly cleared by the body; therefore, there is a need for delivery approaches that (1) target the antibodies to the desired cells, (2) protect the antibodies during their journey and upon entry into the target cells, and (3) allow the antibodies to pass the target cell membrane to enter the cytoplasm. These problems can be solved by mechanically introducing antibodies, antibody derivatives, and proteins/polypeptides into target cells (e.g., via microinjection, electroporation), by directly modifying antibodies, antibody derivatives, and proteins/polypeptides to pass through the cell membrane to enter their target cell by virtue of intrinsic but heterologous properties (e.g., via conjugation to cell-penetrating peptides), and by associating antibodies, antibody derivatives, and proteins/polypeptides to various types of delivery vehicles (e.g., nanoparticles, liposomes, etc.). However, each of these potential solutions has significant limitations that restrict their utility, including (1) microinjection and electroporation are typically used under in vitro conditions, and both exhibit high cytotoxicity and are highly laborious; (2) cell-penetrating peptides promote more efficient delivery, but don't protect antibodies during delivery or allow specific cell targeting; (3) nanoparticles (including liposomes, LNPs, etc.) protect antibodies during delivery and allow some degree of targeting, although they can suffer from significant production demands, low encapsulation efficiency, limited loading capacity, and cytotoxicity. Finally, a shared problematic feature of these delivery systems is endosomal entrapment and subsequent degradation, which precludes the antibody from reaching their cytoplasmic targets intact. An ideal delivery approach would possess several key features, including (1) efficient, stable, and robust prokaryotic encoding and production of the antibodies, (2) high delivery efficiency and intracellularization in vivo, (3) delivery of the antibodies in an active form, (4) targeting to specific cells/tissues, (5) protection of antibody from degradation/clearance, and (6) lack of toxicity and immunogenicity. To date, no delivery platform meets all of these functional requirements.

The efficiency and effectiveness of prokaryotic protein encoding and production prior to delivery can be hindered by a dependence on inefficient or non-optimal naturally occurring prokaryotic regulatory sequences (e.g., promoters and terminators) from other species (i.e., heterologous) or from the host species (i.e., homologous). The activity of heterologous prokaryotic regulatory sequences (either in promoting transcription or terminating transcription) can be suboptimal, thus dampening the therapeutic potential of the delivery system due to limited production of the therapeutic moiety. The use of homologous prokaryotic regulatory sequences may be permissive of homologous recombination events resulting in unintended mutations to chromosomal and/or plasmid DNA sequences and general bacterial genomic instability. Use of highly optimized synthetic regulatory elements, which are regulatory elements rationally and computationally designed to have optimal activity in the host species (e.g., designed for use in E. coli), can overcome these shortcomings. Examples of highly optimized synthetic elements used to regulate the expression of antibodies, antibody derivatives, and proteins/polypeptides inside the bacterial cell described in this invention are presented in Table 1 and Table 2, below. A significant improvement in protein production has been observed by switching to using synthetic promoters that are functional in a prokaryotic system, but not naturally occurring in prokaryotes. By using synthetic prokaryotic promoters, we can dramatically increase bacterial production of these proteins, which in turn improves the entire composition of the system and more proteins are available for delivery.

Taken together, the limitations of current delivery platforms have slowed the translation of intracellular antibody delivery from the research laboratory into clinical use, hampering the potential of antibody-based drugs with intracellular targets. Therefore, there is an urgent need for a more robust production and delivery systems for antibodies, antibody derivatives, antibody fragments, antibody-like molecules and other proteins or polypeptides having limited abilities to cross the cell membrane of a target cell.

Bacterial systems according to aspects of the invention comprise plasmids, or bacteria that are genetically engineered to carry sequences that encode antibodies, antibody derivatives, or proteins/polypeptides whose expression is regulated by synthetic prokaryotic expression sequences (e.g., prokaryotic promoters and terminators that are highly optimized for efficient use in E. coli but are not naturally occurring in the host prokaryotic species, i.e., synthetic) on a plasmid or on the bacterial chromosome. The bacterial cell, therefore, serves as the location for therapeutic moiety production in addition to subsequently serving as the delivery modality to transport the bacterially expressed therapeutic moieties to targeted eukaryotic cells. In one embodiment of this invention, the plasmids, which use prokaryotic promoters to drive expression of the antibodies, antibody derivatives, or proteins/polypeptides, are transformed into the non-pathogenic bacterial cells. In another embodiment, gene(s) encoding the antibodies or antibody derivatives are inserted in the bacterial chromosome with prokaryotic promoters (e.g., optimized, synthetic promoters) to drive their expression. Additionally, the bacterial cells are engineered to be invasive, enabling them to enter eukaryotic cells via receptor-mediated phagocytosis. Small structures, e.g., LNPs, protein conjugates, etc., are taken up by target cells via endocytosis, while larger structures (e.g., bacterial cells) are taken up by phagocytosis. In the context of this bacteria-mediated delivery system, with respect to other delivery platforms, the endosome and phagosome, both membrane-bound compartments, represent roughly equivalent barriers to cytoplasmic delivery. Furthermore, the bacterial cells are genetically modified to enable efficient phagosomal escape of the delivered antibodies, antibody derivatives, or proteins/polypeptides. The combined construct of the bacteria and genes encoding the antibodies, antibody derivatives, and proteins/polypeptides (including the prokaryotic regulatory/expression sequences), whether plasmid-based or chromosomal, constitutes the bacteria-mediated antibody delivery platform by which antibodies, antibody derivatives, and proteins/polypeptides can be produced and intracellularly delivered to targeted eukaryotic cells. Targeting can be directed via the selection of invasion factors, e.g., moieties presented on the surface of the bacterial system, that preferentially bind receptors on the surface of the target cell. An invasion factor can be a factor that facilitates attachment and uptake into a target cell (e.g., invasin protein) and/or a factor that facilities release from a phagosome upon uptake (e.g., lysteriolysin O, LLO). The invasion factors can be produced from genes expressed from the chromosome or plasmid of the bacterial cell. In certain embodiments the bacterium will be engineered to express a particular invasion factor where the invasion factor guides targeting of the delivery platform to a target cell.

In a preferred embodiment, the present invention provides a bacteria-mediated production and delivery platform comprised of invasive, non-pathogenic bacteria for intracellular delivery of antibodies, antibody derivatives, and proteins/polypeptides to eukaryotic cells where the antibodies, antibody derivatives, and proteins/polypeptides produced and delivered are not endogenous to the bacterial vehicle. By “non-pathogenic bacteria”, the bacteria are not capable of causing disease and can be either engineered to be non-pathogenic or are naturally non-pathogenic, although the bacteria may have a cytotoxic or deleterious effect in a target cell as a result of a factor (e.g., polypeptide, antibody derivative) that the bacteria has been engineered to deliver to the target cell.

This bacteria-mediated delivery system uniquely overcomes the deficiencies in other current technologies as described above, offering the following:
(1) High delivery efficiency—upon entry into the eukaryotic cells, the bacteria are engineered to release the antibodies, antibody derivatives, and proteins/polypeptides from the phagosome into the cytoplasm (i.e., phagosomal escape is not a limiting step with our engineered system while endosomal or phagosomal escape of therapeutic modalities continues to be problematic with some delivery systems).
(2) Delivery of the antibodies in an active form—the bacteria themselves produce the antibodies, antibody derivatives, or proteins/polypeptides; therefore, each cell is preloaded with its cargo, thereby allowing for the mature antibodies, antibody derivatives, or proteins/polypeptides to rapidly interact with the target intracellular molecule following delivery to a eukaryotic cell.
(3) Targeting to specific cells and tissues—the bacteria are engineered to be invasive to eukaryotic cells via receptor-mediated phagocytosis. Upon administration to a patient, the bacterial cells traffic to distal tissues or remain in localized tissues, further ensuring high-efficiency, focused delivery to the target tissue. Precise targeting of the bacteria to cells expressing specific surface proteins or chemical moieties is also possible by modifying the invasion factor present on the bacterial cell surface.
(4) Protection of antibodies, antibody derivatives, or proteins/polypeptides from degradation/clearance—the antibodies, antibody derivatives, or proteins/polypeptides are carried inside the bacterial cells, which provide protection from degradation en route to their target eukaryotic cells.
(5) Favorable safety profile—a lack of toxicity and immunogenicity demonstrated in vivo indicates that the bacteria are well-tolerated and are compatible with repeated dosing.
(6) Robust antibody, antibody derivative, and protein/polypeptide expression—the use of highly optimized synthetic regulatory elements designed to be functional in E. coli ensure high expression levels and more reliable termination of the therapeutic antibodies, antibody derivatives, and proteins/polypeptides (specifically polypeptides that are exogenous to, or non-naturally occurring in, the bacterial delivery vehicle).

The present invention offers numerous substantial improvements over the current state of the art. The present invention utilizes bacterial cells containing a prokaryotic expression cassette encoded on a plasmid or on the chromosome to produce and deliver functional antibodies, antibody derivatives, and proteins/polypeptides as cargo to eukaryotic cells. The advantage of using bacteria encoding prokaryotic expression cassettes is that the antibodies are only expressed by the bacteria and are generated by the bacteria prior to delivery, offering greater safety, ability to control dose, and a faster time-to-effect compared to eukaryotic expression systems. Another key advantage of this system is the use synthetic prokaryotic regulatory elements (promoters and terminators) to drive the expression of the invasion factors and antibodies, antibody derivatives, and proteins/polypeptides. This strategy is advantageous as various methodologies have been used to optimize these sequences to ensure high expression levels and more reliable termination of the antibodies, antibody derivatives, and proteins/polypeptides. Once the bacterial cells are taken up and into the cytoplasm of the eukaryotic cell, the plasmid-encoded sequence for the antibody cannot be expressed by the eukaryotic cells due to incompatible sequence requirements.

Another key advantage of the system of the present invention relates to size of the prokaryotic expression plasmid encoding the antibodies, antibody derivatives, and proteins/polypeptides, in which the size of the plasmid backbone is reduced to decrease plasmid-induced burden to the host bacterial cells, thereby increasing the bacterial growth rate. Ideally, the plasmid backbone is less than 10,000 base pairs, in some instances less than 7,000 base pairs, or less than 5,000 base pairs, or less than 3,000 base pairs. Plasmid size can be addressed by transferring certain genes (e.g., the invasion factors) to the chromosome of the bacteria. A reduction in plasmid size can increase copy numbers of the plasmid in the cell and increase expression levels of the plasmid genes.

Based on the deep knowledge of E. coli and general bacterial biology and genetics, an additional benefit of the present invention is that the bacteria's composition can be further modified to comprise additional or new features to enhance the expression efficiency of the antibody, antibody derivative, and protein/polypeptide cargo, enhance the functionality of the antibody, antibody derivative, and protein/polypeptide, enhance the safety profile of the bacterial cell, enhance the immune tolerability profile of the bacterial cell for repeated dosing, and to enhance and/or optimize other aspects of delivery, including the ability to target specific eukaryotic tissues, organs and cells by altering the invasion factors or altering the physical size of the bacterial cell.

For example, the bacterial surface-exposed invasion factor invasin (including fragments or domains of the invasin protein) can be modified via genetic engineering to facilitate bacterial binding to different target proteins expressed on the surface of target eukaryotic cells (e.g., a cell surface receptor such as integrin) or different target chemical moieties expressed on the surface of target eukaryotic cells (e.g., surface accessible N-acetylgalactosamine, GalNAc). As a further example, a chimeric or fusion protein can be created utilizing the transmembrane domain of the invasin polypeptide and the binding domain of a second protein. Examples of the binding domain of a second protein include animal-derived, bacteria-derived, fungus-derived, and/or virus derived heterologous proteins with binding domains (e.g., GalNAc binding proteins, lectins, the group of cell adhesion molecules (CAMs), the group of sulfated glycosaminoglycan (GAG)-binding proteins, selectins, integrins, laminin, cadherins, fibronectin, collagens, thrombospondin, vitronectin, tenascin, apolipoproteins B, E, and A-V, lipoprotein lipase, hepatic lipase, Siglecs, galectins, immunoglobulins, and annexins, FimH, papG, PrsG, Afa-IE, DraA, MrpH, RodA, Mp1, hydrophobins, heat shock protein, CspA, hemagglutinin, neuraminidase, capsid protein, glycoproteins, and envelope protein, among others). This approach allows the bacteria to be targeted to specific cell types, thereby reducing potential off-target effects or undesirable immune stimulation or inducing desirable immune stimulation.

The size of E. coli cells (or other bacterium) could limit their access to target tissues and organs, especially as they traverse the circulatory system and enter small capillaries. Thus, strategies to reduce delivery vehicle size/dimensions can be employed to facilitate delivery to certain target regions that might otherwise provide difficult to access. Mutations can be introduced into the E. coli genome (e.g., in the fabH gene) to reduce the dimensions of the cells.

A third approach is to address limiting bacterial cell components that might prove toxic or otherwise detrimental to the target cell. Some target cell types might be more sensitive to residual bacterial components (e.g., lipopolysaccharide, LPS) deposited after invasion and cargo delivery to a eukaryotic cell, possibly leading to cytotoxicity and cell death. In some cases, this cell death might be traceable to a single pathway (e.g., caspase-mediated cell death). The bacteria can be genetically modified to deliver additional heterologous factors (e.g., caspase-inhibitory viral proteins) that ameliorate such effects. This approach offers the additional benefit of increasing delivery efficiency by preserving the health of the invaded eukaryotic cells and preventing unfavorable cytotoxicity. Additionally, the bacteria can be genetically modified to limit pathogenicity and immune stimulation by altering, modifying, mutating, or removing bacterial virulence factors (e.g., mutating the msbB gene resulting in LPS that lacks the myristoyl fatty acid moiety of the lipid A).

In some applications, the bacteria must traverse endothelial cell layers (e.g., to exit the circulation through capillary walls) to reach the target cells or tissues. Bacteria have various levels of ability to traverse such barriers and these abilities can be conferred by a single or a few bacterially-encoded proteins. To increase delivery efficiency, such proteins (“entry proteins”) can be borrowed from one bacterial species or strain to introduce this ability into the delivery strain. For example, some strains of E. coli and other bacteria readily traverse endothelial layers, and the genes encoding these proteins could be introduced into a delivery bacterial strain to enhance tissue biodistribution throughout multiple cell layers, thereby increasing delivery efficiency. Examples of such entry proteins include FimH, OmpA, IbeA, IbeB, IbeC, Opc, PilA, PilB, LOS, Lmb, FbsA, IagA, Vsp1, OspA, 70-kDa PBP, enolase, Isc1, Yps3p, Stx, type 3 secretion system-injected factors, EspF, Map, EspG).

Approaches for generating antibody conjugates are technically complex and the conjugated elements can have toxic effects. The presently described bacteria-mediated system overcomes these issues as it does not require conjugation of the therapeutic moiety to a potentially toxic compound or packaging in particles (e.g., LNPs) that can also exhibit toxicity. Furthermore, the present invention eliminates complex manufacturing steps, e.g., chemical conjugation, production of and packaging into nanoparticles, etc. A prominent problem with other systems, especially those involving free antibodies conjugated to a cell penetrating moiety, is the challenge of protecting the antibody from degradation upon introduction into the body (particularly in the circulatory system). The bacteria-mediated system taught herein overcomes this challenge as the therapeutic moiety is protected within the bacterial cell until it reaches and is delivered into the target host cells.

The present invention provides a bacteria-mediated production and delivery platform comprised of invasive, non-pathogenic bacteria for intracellular delivery of antibodies, antibody derivatives, and proteins/polypeptides to eukaryotic cells. The bacteria can contain a prokaryotic expression cassette encoding the antibody, antibody derivative, or protein/polypeptide cargo. More specifically, this bacterial delivery system is comprised of at least one antibody, antibody derivative, or protein/polypeptide nucleotide coding sequence that is integrated into the bacterial chromosome or expressed from at least one plasmid within the bacterium. The bacterium used in the production and delivery system of the invention can be one from a variety of species including Lactobacillus spp., Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Leishmania spp. and Erysipelothrix spp., Shigella spp., Listeria spp., Rickettsia spp., Acetoanacrobium spp., Acrococcaceae spp., Carnobacteriaceae spp., Enterococcace spp., Leuconostocacease spp., Streptococcaceae spp., Bifidobacterium spp., and bacteria with generally regarded as safe (“GRAS”) status. In a preferred aspect, the bacterium is Escherichia coli. For controlled and enhanced transcription of the nucleic acid encoding the antibody, antibody derivative, and protein/polypeptide, the nucleic acid coding sequence is controlled by a synthetic prokaryotic promoter and synthetic prokaryotic terminator. In addition, the bacterium can deliver antibody, antibody derivative, or protein/polypeptide into the cytoplasm of a eukaryotic cell by expressing invasion factors (e.g., invasin, either complete or a fragment thereof, HylA, HlyE, influenza HA-1) that facilitate entry into the eukaryotic cells via receptor-mediated phagocytosis followed by efficient phagosomal escape of the delivered antibody, antibody derivative, or protein/polypeptide. The antibody, antibody derivative, or protein/polypeptide that is encoded and delivered by the bacterium can modify the activity or function of at least one intracellular factor located inside the cytoplasm of a eukaryotic cell. The delivered antibody, antibody derivative, or protein/polypeptide can be agonistic or antagonistic to intracellular pathways by interacting with specific target molecules. Example intracellular pathways include targeting and/or inactivating replication and survival mechanisms of infectious pathogens, including intracellular bacterial pathogens, protozoal pathogens, viruses, and fungal pathogens. Other examples of therapeutic applications include producing and delivering antibodies, antibody derivatives, and proteins/polypeptides that target, and/or inactivate intracellular factors associated with cancer, metabolic disease, neurodegeneration, protein overexpression/aggregation disorders (e.g., Alzheimer's and Parkinson's diseases, amyotrophic lateral sclerosis, Lewy body dementia, frontotemporal dementia, Huntington's disease, amyloid transthyretin cardiomyopathy, type-2 diabetes, and any other type of amyloidosis), inflammatory disorders, and autoinflammatory disease. As a cancer application, the delivery of antibodies can offer the advantage of being able to target multiple isoforms of a protein or proteins carrying a spectrum of mutations. For example, mutations in the proto-oncogene KRAS are present in many human cancer types; however, the range of mutations is diverse. This diversity, in addition to other structural features of KRAS protein, makes the discovery of therapeutic KRAS inhibitors exceedingly difficult. Due to their size and recalcitrance to subtle structural features that do not affect the direct interaction epitope, a single antibody or antibody derivative (e.g., nanobodies) can be used as a robust inhibitor of a range of proteins, including multiple RAS isoforms (e.g., KRAS4A, KRAS4B, HRAS, and NRAS) and many mutated KRAS proteins. This inhibitory effect, when coupled with a robust delivery platform such as that described here, open new opportunities for the development of both RAS-targeted cancer therapeutics as well as therapeutics that benefit from broad target flexibility. Additional therapeutic applications include producing and delivering polypeptides that can serve as antigens to stimulate an antibody response in vaccine applications, and/or polypeptides, such as enzymes or proteins, that supplement or replace endogenous polypeptides whose production is dysregulated or altered due to a disease state or genetic disorder. There are also diagnostic applications for this invention, including applications related to diagnostic intracellular imaging processes. For example, the protein being delivered could be an affibody or serve as an intracellular probe to detect intracellular targets indicative of disease. This includes, for example, delivery of nanoflares (probe-like molecules) that can be used to detect intracellular targets, such as mRNAs coding for genes over-expressed in cancer (epithelial-mesenchymal transition, oncogenes, thymidine kinase, telomerase, etc.), intracellular levels of ATP, pH values and inorganic ions. This would also allow for diagnosing disease and/or elucidating intracellular processes within living cells in real time.

Therapeutic applications (for humans and animals) include, but are not limited to:

Virology: antibodies, antibody derivatives, and proteins/polypeptides can be produced and delivered using the present invention to target and inactivate essential proteins required for intracellular viral replication. This inactivation occurs, for example, via disruption of proper protein folding, blocking allosteric structural changes, blocking active sites, blocking binding sites for interacting proteins, or blocking binding sites of co-factors, e.g., ATP, GTP, etc. by the antibody, antibody derivative, or proteins/polypeptides. Possible applications include inhibition of HIV replication via inhibition of HIV integrase activity; inhibition of norovirus replication; inhibition of influenza A replication; inhibition of Ebola virus replication; inhibition of hepatitis virus replication (all types); inhibition of coronavirus replication (all types).

Bacteriology: antibodies, antibody derivatives, and proteins/polypeptides can be produced and delivered using the present invention to target/inactivate essential proteins required for the intracellular lifestyle of intracellular bacterial pathogens, e.g., proteins required for nutrient uptake from the bacterial host (siderophores, etc.). Possible applications include elimination of Ehrlichia chaffeensis, Staphylococcus aureus, Chlamydia, Rickettsia, Coxiella, Mycobacteria spp., Brucella, Legionella, Nocardia, Neisseria, Rhodococcus equi, Yersinia, Francisella tularensis, and Bartonella henselae.

Protozoans: antibodies, antibody derivatives, and proteins/polypeptides can be produced and delivered using the present invention to target/inactivate essential proteins required for the intracellular lifestyle of intracellular protozoal pathogens, e.g., proteins required for nutrient uptake from the protozoan host. Possible applications include elimination of Trypanosomatids (e.g., Leishmania spp., Trypanosoma spp.), Apicomplexans (e.g., Plasmodium spp., Toxoplasma gondii, Cryptosporidium parvum).

Mycology: antibodies, antibody derivatives, and proteins/polypeptides can be produced and delivered using the present invention to target/inactivate essential proteins required for the intracellular lifestyle of intracellular fungal pathogens, e.g., proteins required for nutrient uptake from the fungal host. Possible applications include elimination of Pneumocystis jirovecii, Histoplasma capsulatum, Candida albicans, and Cryptococcus neoformans.

Antibodies, antibody derivatives, and proteins/polypeptides can also be generated and delivered using the present invention to target/inactivate proteins in intracellular signaling pathways that have therapeutic potential. Examples of pathways, target proteins, and associated therapeutic areas include:
(1) Apoptosis/cell death/cell proliferation, and associated disorders, e.g., PI3K, Akt, HIF1A, p53, Blc2, Bcl-XL, Bcl-w, BRUCE, MCL-2, XIAP, cIAP1, C-IAP2, NAIP, Livin, survivin (BIRC5), cancer.
(2) Warburg effect-related proteins and associated disorders (e.g., GLUT1, GLUT3, PDK1, PDK2, MAGL, HK, PKM2, LDHA, G6PD, MCT1, PKB; cancer, metabolic disorders).
(3) Nutrient signaling proteins and associated disorders (e.g., mTor or any protein found in the mTORC1 or mTORC2 complexes; cancer; reduction of neurodegeneration-associated cognitive decline; metabolic disease).
(4) Wnt pathway and associated disorders (e.g., Wnt, Frizzled, LRP5/6, etc.; cancer).
(5) NFkB pathway and associated disorders (e.g., TNF-α, JAK1, etc.; cancer; inflammatory disorders).
(6) Notch pathway and associated disorders (e.g., γ-secretase, Notch 1, Notch 2, Notch 3; cancer).
(7) Sonic Hedgehog pathway and associated disorders (e.g., GLI1, GLI2, SMO; cancer).
(8) TLR signaling and associated disorders (TLR1, TLR2, TLR3, TLR4, TLR5, TLR6, TLR7, TLR8, TLR9, TLR10, TLR11, TLR12, TLR13; leukemia, colon cancer, myelodysplastic syndrome, autoinflammatory diseases, inflammatory diseases).

### Example: Delivery of a Survivin-Inactivating Single-Domain Antibody (Nanobody) Inhibits Cancer Cell Proliferation

In eukaryotic cells, survivin/BIRC5 (a member of the inhibitor of apoptosis protein family) inhibits apoptosis (i.e., programmed cell death) by blocking caspase activation. Survivin is highly expressed in many proliferating human cancer cell types and is absent in terminally differentiated, post-mitotic cells; therefore, survivin inhibition preferentially affects proliferating cancer cells.

Cancer cells are typified by uncontrolled proliferation, often due to a defect in apoptosis. A potent therapeutic approach to limit cancer/tumor growth is to increase apoptosis, particularly that triggered by excessive DNA damage.

Disruption of survivin function leads to increased apoptosis and decreased cancer cell proliferation. These features highlight survivin as a potential cancer therapeutic target that would allow discrimination between cancer cells and normal cells, and indeed, survivin has been studied as a cancer therapeutic for over 20 years. Unfortunately, multiple clinical trials of survivin-based therapeutics have failed. A persistent challenge is delivery of the survivin-inhibiting moiety to cancer cells in the same cellular compartment (the cytoplasm) in which survivin exerts its function. The bacteria-based delivery system described herein will overcome this challenge by delivering a bacterially-encoded and expressed single-domain antibody (nanobody) to the cytoplasm of targeted cells that specifically inhibits survivin.

When the amount of DNA damage accumulated by a cell reaches a threshold, the apoptotic cascade is initiated to remove the cell from the population via apoptosis or programmed cell death. However, survivin inhibits cancer cell death by effectively increasing the threshold that must be reached for apoptotic initiation. Most chemotherapeutic drugs used to treat cancer are toxic not only to tumor cells, but also to normal tissue; thus, systemic chemotherapy can have profound off-target effects. Therefore, it is advantageous to use the lowest efficacious dose to minimize these undesired effects. Many chemotherapeutic agents activate apoptosis in cells by inducing sufficient DNA damage to cross the above-mentioned threshold. Survivin is a barrier to apoptosis activation due to its effect on this threshold. Accordingly, the dosage of a chemotherapeutic agent must be sufficiently high to overcome it. Inactivation of survivin, for example with a nanobody, could reduce the threshold for apoptosis activation, thereby reducing the efficacious dose of a chemotherapeutic agent, possibly reducing off-target effects without compromising its anti-tumorigenic effect. In this approach, a survivin-inactivating nanobody produced and delivered using the bacterial system of the present invention would function as a chemotherapeutic enhancer or, in some cases, a chemotherapeutic adjuvant therapy.

Here, we confirm the successful delivery of a functional, bacterially expressed survivin-inhibiting nanobody (nb) protein (i.e., anti-survivin nb) using the herein-described invasive bacterial delivery system, which consists of diaminopimelic acid auxotrophic Escherichia coli (FEC21), by showing that the proliferation of adenocarcinoma human alveolar basal epithelial cells (A549 cells, a common model cancer cell line) is slowed upon treatment of the cells with invasive bacteria that deliver the anti-survivin nb to the cancer cell cytoplasm. These results demonstrate the potential of this bacterial delivery system as a novel approach for developing cancer therapeutics based on the delivery of protein factors that interfere with cancer cell function.

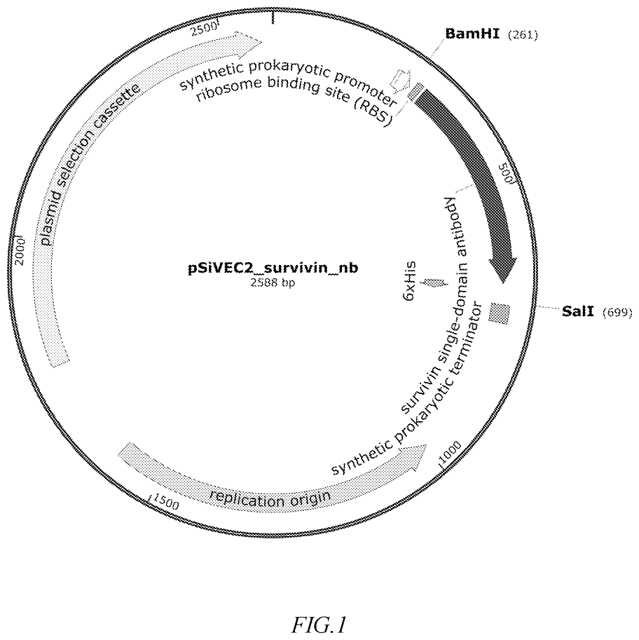

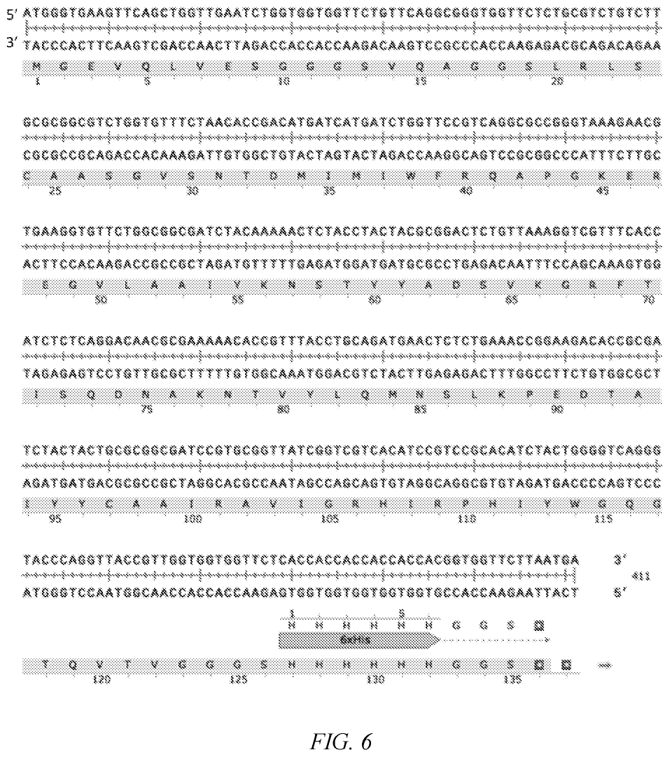

An E. coli-optimized, anti-survivin nb (FIG. 6, boxed asterisks indicate termination codons) sequence was cloned into empty pSiVEC2 vector to generate the pSiVEC2_survivin_nb plasmid (FIG. 1). The cloned sequence also encodes a translationally fused 6×His affinity tag on the C-terminus of the nb. In pSiVEC2_survivin_nb, prokaryotic expression of the anti-survivin nb protein is controlled by a prokaryotic promoter (i.e., a promoter that is only active in bacterial cells). Therefore, the bacteria both produce (transcribe and translate) and deliver the anti-survivin nb to eukaryotic cells.

The pSiVEC2_survivin_nb was transformed into FEC21 E. coli bacteria to generate the strain FEC21/pSiVEC2_survivin_nb. The FEC21 bacteria were additionally engineered to be invasive to eukaryotic cells via chromosomal integration of the inv and hlyA genes for invasin- and receptor-mediated phagoctysosis and HylA-mediated endosomal release, respectively. FEC21 cells transformed with pSiVEC2_survivin_nb were plated onto brain heart infusion (BHI) agar containing appropriate antibiotics for selection. FEC21/pSiVEC2_survivin_nb clones were frozen at −80° C. in 20% glycerol.

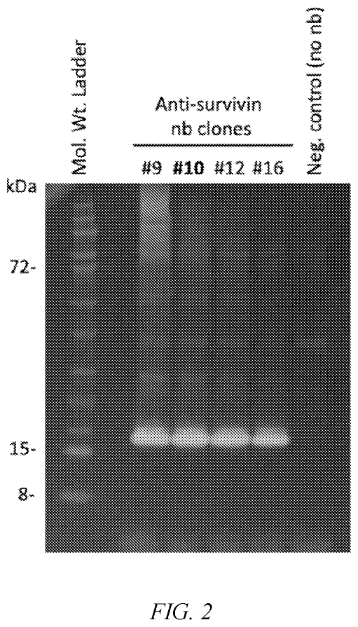

Bacterial expression of the anti-survivin nb was confirmed via western blotting of denatured protein samples of FEC21/pSiVEC2_survivin_nb cells using an anti-6×His tag antibody (diluted 1:500), which binds to the C-terminal 6×His-tag fused to the anti-survivin nb. FIG. 2 shows the results of this western blot for four independent clones of FEC21/pSiVEC2_survivin_nb (labeled #9, #10, #12, and #16). A robust band representing a protein of approximately 15 kDa, which was absent in the negative control lane, confirms strong bacterial expression of the anti-survivin nb.

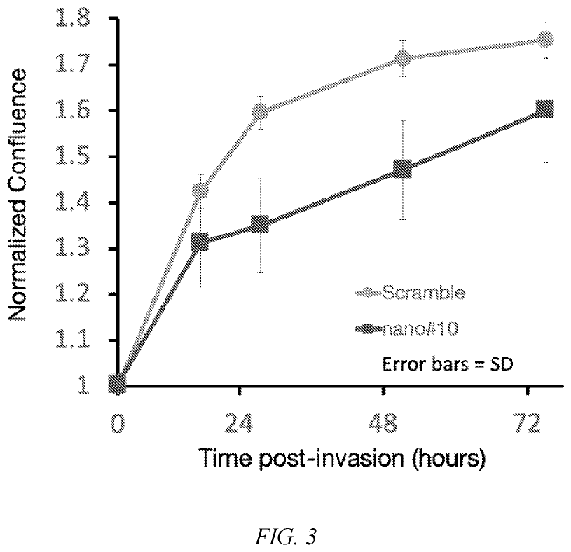

A standard invasion assay (i.e., when the FEC21 bacteria are incubated with mammalian cells and invade the mammalian cells to become intracellularized) was used to determine whether delivery of the bacterially expressed anti-survivin nb by invasive FEC21 (FIG. 3, clone #10) bacteria could reduce the proliferation of alveolar basal epithelial cells (A549), a common cancer cell line. A549 cells were maintained in Dulbecco's modified Eagle's medium (DMEM) supplemented with 10% fetal bovine serum, 2 mM GlutaMAX, 100 U/mL penicillin, and 100 g/mL streptomycin at 37° C. with 5% CO₂ incubation. The invasive bacteria (by virtue of encoding inv and hlyA) enter A549 cells via RME to deliver their bacterially encoded and expressed cargo (i.e., the anti-survivin nb). This invasion assay includes the following steps: A549 cells are seeded at a fixed concentration into black 24-well plates. On the day of bacterial invasion, two bacterial stocks are thawed: 1) FEC21/pSiVEC2_survivin_nb (clone #10) and 2) FEC21/pSiVEC2_Scramble (an invasive negative control bacterial strain transformed with a plasmid carrying a non-coding scramble sequence). The bacterial cells are centrifuged and resuspended in DMEM (−) (serum- and antibiotic-free, high-glucose DMEM) at an absorbance 600 (A600) of 0.004. The A549 cells are washed in DMEM (−) to remove antibiotics and are incubated for 2 hours (37° C. with 5% CO₂) with 0.5 mL of each bacterial suspension and subsequently rinsed with DMEM to remove residual bacteria that did not invade the A549 cells. A Nexcelom Celigo instrument (in the brightfield channel) was used to measure cell confluence (an indicator of cell proliferation) at 0, 18, 28, 52, and 76 hours post invasion.

FIG. 3 shows that A549 cells receiving the anti-survivin nb via the herein-described bacterial delivery system have a sustained reduction in proliferation compared with cells that received the scramble sequence (no-nanobody control). Together, these results demonstrate that the invasive FEC21 bacterial delivery platform can both express and deliver functional anti-survivin nb protein to lung epithelial cells to limit their proliferation. N=6 biological replicates per timepoint per condition. The cell confluence at each timepoint was normalized to the starting confluence of that condition. Data shown are the mean±standard deviation.

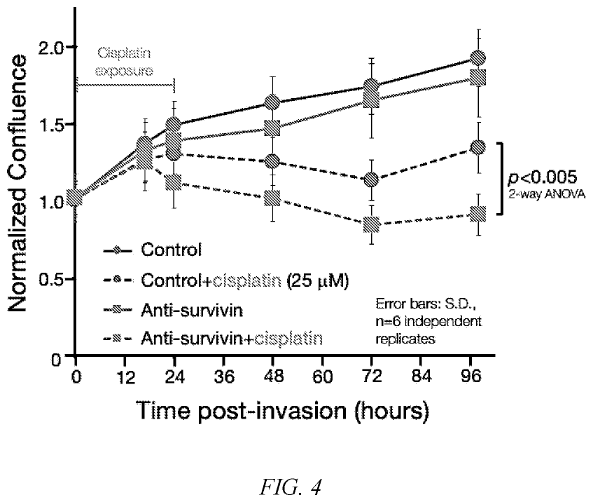

FIG. 4 demonstrates the use of the same anti-survivin nb as a chemotherapeutic adjuvant. A549 cells were treated with invasive bacteria expressing the anti-survivin nanobody as described above (FEC21/pSiVEC2_survivin_nb clone #10) and then exposed to the common chemotherapeutic cisplatin (25 μM) for the first 24 hours after bacterial treatment, which activates apoptosis in cancer cells by inducing DNA damage. Cell confluence was measured, analyzed, and plotted as described above. As shown in the plot, delivery of the anti-survivin nb via the bacteria-mediated delivery system described herein enhances the effect of cisplatin treatment on cell proliferation, establishing FEC21/pSiVEC2_survivin_nb as a chemotherapeutic enhancer, i.e., delivery of the anti-survivin nb increases the efficacy of a given dose of cisplatin. Robust statistical significance (p<0.005) was assessed via 2-way ANOVA.

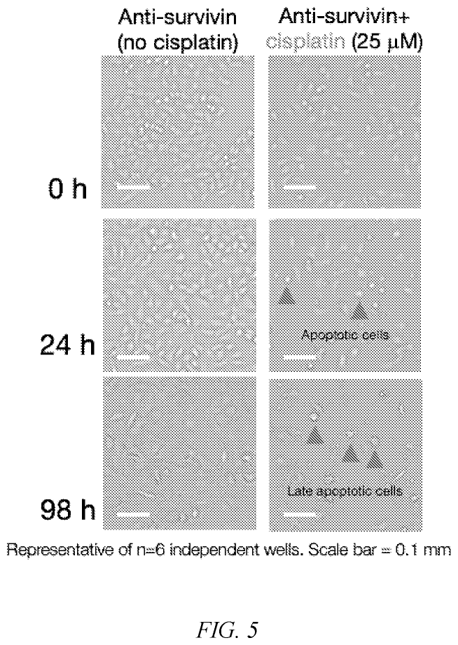

FIG. 5 is a set of images that shows the increased rate of apoptosis in the cells analyzed for the production of FIG. 4.

### REFERENCES

Pellis, M.; Pardon, E.; Zolghadr, K.; Rothbauer, U.; Vincke, C.; Kinne, J.; Dierynck, I.; Hertogs, K.; Leonhardt, H.; Messens, J.; Muyldermans, S.; Conrath, K. A Bacterial-Two-Hybrid Selection System for One-Step Isolation of Intracellularly Functional Nanobodies. Archives of Biochemistry and Biophysics 2012, 526, 114-123.
Koromyslova, A. D.; Hansman, G. S. Nanobody Binding to a Conserved Epitope Promotes Norovirus Particle Disassembly. Journal of Virology 2014, 89, 2718-2730.
Cavallari, M. Rapid and Direct VHH and Target Identification by Staphylococcal Surface Display Libraries. International. Journal of Molecular Sciences 2017, 18, 1507.
M. Winslow, Eric Yager, Konstantin Shilo, Erin Volk, Andrew Reilly, Frederick K. Chu. Antibody-Mediated Elimination of the Obligate Intracellular Bacterial Pathogen Ehrlichia chaffeensis during Active Infection. Gary Infection and Immunity April 2000, 68 (4) 2187-2195; DOI: 10.1128/IAI.68.4.2187-2195.2000
Wenqing Zhang, Mingqun Lin, Qi Yan, Khemraj Budachetri, Libo Hou, Ashweta Sahn, Hongyan Liu, Nien-Ching Han, Jeffrey Lakritz, Dehua Pei, Yasuko Rikihisa. An intracellular nanobody targeting T4SS effector inhibits Ehrlichia infection. Proceedings of the National Academy of Sciences May 2021, 118 (18) e2024102118; DOI: 10.1073/pnas.2024102118
Ye Wang, Haochen Wang, Lei Wei, Shuailin Li, Liyang Liu, Xiaowo Wang, Synthetic promoter design in Escherichia coli based on a deep generative network, Nucleic Acids Research, Volume 48, Issue 12, 9 Jul. 2020, Pages 6403-6412, https://doi.org/10.1093/nar/gkaa325
Chen Y J, Liu P, Nielsen A A, Brophy J A, Clancy K, Peterson T, Voigt C A. Characterization of 582 natural and synthetic terminators and quantification of their design constraints. Nat Methods. 2013 July; 10 (7): 659-64. doi: 10.1038/nmeth.2515. Epub 2013 Jun. 2. PMID: 23727987.
Andrew J Hudson, Hans-Joachim Wieden, Rapid generation of sequence-diverse terminator libraries and their parameterization using quantitative Term-Seq, Synthetic Biology, Volume 4, Issue 1, 2019, ysz026, https://doi.org/10.1093/synbio/ysz026

### Definitions

As used in the application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or”, and “all or any other combination of the elements connected by said term”.

The term “about” or “approximately” as used herein means within 20%, preferably within 10%, and more preferably within 5% of a given value or range.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

The term “administration” and variants thereof (e.g., “administering” a compound) in reference to a compound of the invention means introducing the compound into the system of the subject in need of treatment. When a compound of the invention is provided in combination with one or more other active agents, “administration” and its variants are each understood to include concurrent and sequential introduction of the compound and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to treating cancer, an effective amount comprises an amount sufficient to prevent clinical disease or to reduce the severity of the disease as evidenced by clinical disease, clinical symptoms. In some embodiments, an effective amount is an amount sufficient to delay onset of clinical illness and/or symptoms or to prevent the disease. An effective amount can be administered in one or more doses.

As used herein, “treatment” refers to obtaining beneficial or desired clinical results. Beneficial or desired clinical results include, but are not limited to, any one or more of: alleviation of one or more symptoms, diminishment of extent of disease, stabilized (i.e., not worsening) state of disease or disease symptoms, preventing or delaying spread of the disease, preventing, delaying or slowing of disease progression, and/or maintain weight/weight gain. The methods of the invention contemplate any one or more of these aspects of treatment.

A “pharmaceutically acceptable” component is one that is suitable for use with humans and/or animals without undue adverse side effects (such as toxicity, irritation, and allergic response) commensurate with a reasonable benefit-to-risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

As used herein, a “promoter” or “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence a transcription initiation site will be found, as well as protein binding domains responsible for the binding of RNA polymerase. Various promoters, including inducible promoters, may be used to drive the vectors as described in the present invention.

A promoter can be a constitutively active promoter (i.e., a promoter that is constitutively in an active (“ON”) state), or it may be an inducible promoter (i.e., a promoter whose state, active (“ON”) or inactive (“OFF”), controlled by an external stimulus (e.g., the presence of a particular temperature, compound, or protein).

As used herein, a synthetic prokaryotic promoter is a non-naturally occurring DNA sequence rationally designed to have transcription-promoting (i.e., containing −35, −10, and UP sequences that recruit RNA polymerase) activity in a bacterial cell. As used herein, a synthetic prokaryotic terminator is a non-naturally occurring DNA sequence rationally designed to have transcription-terminating (i.e., containing sequences that terminate transcription via intrinsic or Rho-dependent mechanisms) activity in a bacterial cell. Further, a “synthetic” DNA sequence is a DNA sequence that does not exist in nature and has rather been artificially created (e.g., via bioinformatic or in silico techniques) for a specific purpose.

Antibodies, or immunoglobulins (Ig), are large, Y-shaped proteins with a size of approximately 150 to 160 kDa, which are composed of two heavy protein chains and two light protein chains, and even smaller than Fab fragments (approximately 50 kDa, one light chain and half a heavy chain) and single-chain variable fragments (approximately 25 kDa, two variable domains, one from a light and one from a heavy chain).

As used herein, the term “antibody derivative” is a polypeptide whose amino acid chain (i.e., primary sequence) adopts a tertiary folding structure that shares structural homology to any domain or subdomain found in an antibody and/or whose structure allows the polypeptide to interact with another cellular component.

An “affibody”, or affibody molecule is small, robust protein that engineered to bind to a target protein (e.g., antigen) or peptides with high affinity. Affibody binding is analogous to that of a monoclonal antibody, thus making affibodies antibody mimetics. Affibodies can be used for molecular recognition in diagnostic and therapeutic applications.

A “nanobody”, or single-domain antibody (sdAb), is an antibody fragment consisting of a single monomeric variable antibody domain. Like a whole antibody, a nanobody is able to bind selectively to a specific antigen. Nanobodies have a molecular weight of only approximately 12-15 kDa, making nanobodies/single-domain antibodies much smaller than common antibodies (150-160 kDa) which are composed of two heavy protein chains and two light chains, and even smaller than Fab fragments (approximately 50 kDa, one light chain and half a heavy chain) and single-chain variable fragments (approximately 25 kDa, two variable domains, one from a light and one from a heavy chain).

A V_{HH} antibody, or nanobody, is the antigen-binding fragment of heavy chain-only antibodies.

As used herein, the term “invasive” when referring to a microorganism, e.g., a bacterium or bacterial therapeutic particle (BTP), refers to a microorganism that is capable of delivering at least one molecule, e.g., an antibody, antibody derivative, and protein/polypeptide, to a target cell. An invasive microorganism can be a microorganism that is capable of traversing a cell membrane, thereby entering the cytoplasm of said cell, and delivering at least some of its content, e.g., antibodies, antibody derivatives, and proteins/polypeptides, into the target cell. The process of delivery of the at least one molecule into the target cell preferably does not significantly modify the invasion apparatus.

As used herein, the term “cellular targeting factor” is a moiety expressed on the surface of the bacterial cell that allows the bacterial cells to specifically interact with a specific type or class of eukaryotic cell (i.e., the target cell).

As used herein, the term “domain” or “protein domain” refers to a sequence of amino acids within the protein's polypeptide chain that folds independently of the rest of the protein and is self-stabilizing. A domain folds into a compact three-dimensional structure.

As used herein, the term “endogenous” or “endogenously expressed” when referring to a antibody, antibody derivative, and protein/polypeptide means the antibody, antibody derivative, and protein/polypeptide is naturally produced by the organism.

As used herein, the term “transkingdom” refers to a delivery system that uses bacteria (or another invasive microorganism) to generate antibodies, antibody derivatives, and proteins/polypeptides, and deliver the antibodies, antibody derivatives, and proteins/polypeptides intracellularly (i.e., across kingdoms: prokaryotic to eukaryotic, or across phyla: invertebrate to vertebrate) within target tissues to modulate the activity of a target molecule in the eukaryotic cell without host genomic integration. The bacteria will be “non-pathogenic” regardless of the presence or absence of the particular expression cassette carrying the encoded antibodies, single V_{HH} antibody domains, nanobodies, and/or other antibody derivatives (or other therapeutic nucleic acid). However, in some instances the cargo being carried and delivered by the bacteria can have cytomodulatory or cytotoxic effects on the recipient eukaryotic cells. “non-pathogenic bacteria”, the bacteria are not capable of causing disease and can be either engineered to be non-pathogenic or are naturally non-pathogenic in the absence of a specific factor (e.g., polypeptide, antibody derivative) that the delivery system was engineered to deliver to the target cell.

Invasive microorganisms include microorganisms that are naturally capable of delivering at least one molecule to a target cell, such as by traversing the cell membrane, e.g., a eukaryotic cell membrane, and entering the cytoplasm, as well as microorganisms which are not naturally invasive and which have been modified, e.g., genetically modified, to be invasive. In another preferred embodiment, a microorganism that is not naturally invasive can be modified to become invasive by linking the bacterium or BTP to an “invasion factor”, also termed “entry factor” or “cytoplasm-targeting factor”. As used herein, an “invasion factor” is a factor, e.g., a protein or a group of proteins which, when expressed by a non-invasive bacterium or BTP, render the bacterium or BTP invasive. As used herein, an “invasion factor” provides targeting, uptake, and/or export functions and can be engineered as a chimeric factor (i.e., a recombinant protein encoded by a heterologous gene sequence fused in frame to a fragment of another invasion factor or subunit thereof). As used herein, an “invasion factor” is encoded by a “cell-targeting gene”. Invasive microorganisms have been generally described in the art, for example, U.S. Pat. Pub. Nos. US20100189691 A1 and US20100092438 A1 and Xiang, S. et al., Nature Biotechnology 24, 697-702 (2006). Each of which is incorporated by reference in its entirety for all purposes.

In a preferred embodiment the invasive microorganism is E. coli, as taught in the examples of the present application. However, it is contemplated that additional microorganisms could potentially be adapted to perform as transkingdom delivery vehicles for the delivery of gene-editing cargo. These non-virulent and invasive bacteria and BTPs would exhibit invasive properties, or would be modified to exhibit invasive properties, and may enter a host cell through various mechanisms. In contrast to uptake of bacteria or BTPs by professional phagocytes, which normally results in the destruction of the bacterium or BTP within a specialized lysosome, invasive bacteria or BTP strains have the ability to invade non-phagocytic host cells. Naturally occurring examples of such intracellular bacteria are Yersinia, Rickettsia, Legionella, Brucella, Mycobacterium, Helicobacter, Coxiella, Chlamydia, Neisseria, Burkolderia, Bordetella, Borrelia, Listeria, Shigella, Salmonella, Staphylococcus, Streptococcus, Porphyromonas, Treponema, and Vibrio, but this property can also be transferred to other bacteria or BTPs such as E. coli, Lactobacillus, Lactococcus, or Bifidobacteriae, including probiotics through the transfer of invasion-related genes (P. Courvalin, S. Goussard, C. Grillot-Courvalin, C.R. Acad. Sci. Paris 318, 1207 (1995)). Factors to be considered or addressed when evaluating additional bacterial species as candidates for use as transkingdom delivery vehicles include the pathogenicity, or lack thereof, of the candidate, the tropism of the candidate bacteria for the target cell, or, alternatively, the degree to which the bacteria can be engineered to deliver gene-editing cargo to the interior of a target cell, and any synergistic value that the candidate bacteria might provide by triggering the host's innate immunity.

The methods of administering these improved bacterial delivery vehicles include intraperitoneal and intravenous dosing for systemic delivery, intrathecal for CNS delivery, intramuscular injection for dosing to the skeletal muscle, intranasal dosing to nasal cavity for local action, aerosolization for upper and lower respiratory targeting, absorption in the oral cavity for buccal delivery, ingestion for GI adsorption, application to delicate genital mucosal epithelium, and topical administration for ocular delivery. These improved delivery vehicles could be used to prevent and/or treat a wide range of diseases (infectious, allergic, cancerous, genetic, and immunological) in a wide range of species (human, avian, swine, bovine, canine, equine, feline).

The term “administration” and variants thereof (e.g., “administering” a compound) in reference to a compound of the invention means introducing the compound into the system of the subject in need of treatment. When a compound of the invention is provided in combination with one or more other active agents (e.g., a cytotoxic agent, etc.), “administration” and its variants are each understood to include concurrent and sequential introduction of the compound and other agents.

A “subject” is any multi-cellular vertebrate organism, such as human and non-human mammals (e.g., veterinary subjects). In one example, a subject is known or suspected of having an infection or other condition that is life-threatening or impairs the quality of life.

The terms “treating” and “treatment” as used herein refer to the administration of an agent or formulation (e.g., bacterium) of the invention to a clinically symptomatic subject afflicted with an adverse condition, disorder, or disease, so as to affect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

As used herein, a “biologically active peptide” is a peptide that has an effect on a living organism, tissue, cell, or biochemical process. A biologically active peptide can be grafted onto an Fc domain or other antibody domain (e.g., a peptibody) of the antibody or antibody derivative to deliver the function of the biologically active peptide, along with the antibody or antibody derivative to which it is grafted, to a target cell. The effect of the biologically active peptide can be that of an antioxidant, antimicrobial, immunomodulatory, cytomodulatory, and/or metabolism-altering properties.

Invasive bacteria containing the antibody, antibody derivative, and protein/polypeptide can be introduced into a subject by intravenous, intramuscular, intradermal, intraperitoneally, peroral, intranasal, intraocular, intrarectal, intravaginal, intraosseous, oral, immersion and intraurethral inoculation routes. The amount of the invasive bacteria of the present invention to be administered to a subject will vary depending on the species of the subject, as well as the disease or condition that is being treated. For example, a dosage could be approximately 10³ to 10¹¹ viable organisms, preferably approximately 10⁵ to 10⁹ viable organisms per subject. The invasive bacteria or BTPs of the present invention are generally administered along with a pharmaceutically acceptable carrier and/or diluent. In some instances, the invasive bacteria or BTPs of the present invention are formulated as a dry powder, lyophilized, or freeze dried.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

For administration by inhalation, the pharmaceutical compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the composition, e.g., bacteria, and a suitable powder base such as lactose or starch.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water.

The invasive bacteria containing the antibodies, antibody derivatives, and proteins/polypeptides to be introduced can be used to infect animal cells that are cultured in vitro, such as cells obtained from a subject. These in vitro-infected cells can then be introduced into animals, e.g., the subject from which the cells were obtained initially, intravenously, intramuscularly, intradermally, or intraperitoneally, or by any inoculation route that allows the cells to enter the host tissue. When delivering antibodies, antibody derivatives, and proteins/polypeptides to individual cells, the dosage of viable organisms administered will be at a multiplicity of infection ranging from approximately 0.1 to 10⁶, preferably approximately 10² to 10⁴ bacteria per cell.

Kits for practicing the methods of the invention are further provided. By “kit” is intended any manufacture (e.g., a package or a container) comprising at least one reagent, e.g., a pH buffer of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

In an advantageous embodiment, the kit containers may further include a pharmaceutically acceptable carrier. The kit may further include a sterile diluent, which is preferably stored in a separate additional container. In another embodiment, the kit further comprising a package insert comprising printed instructions directing the use of a combined treatment of a pH buffer and the anti-pathogen agent as a method for treating and/or preventing disease in a subject. The kit may also comprise additional containers comprising additional anti-pathogen agents (e.g., amantadine, rimantadine, and oseltamivir), agents that enhance the effect of such agents, or other compounds that improve the efficacy or tolerability of the treatment.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

TABLE 1

| Synthetic Promoter Sequence | |
|---|---|
| (5′-3′) | Name |
| AATTCTTTTTGACGCATAAGTTGTGAA | WGAN-GP_sfgfp_ |
| ATCGTGTAATTTCTGCTATG | round1_13 |
| CTAGGTTCA [SEQ ID NO. 1] | |
| AATTCTGTCAGGTTTTGAACTTGAAGA | WGAN-GP_sfgfp_ |
| AGAAGTGGTGTGTGGCTAT | round1_22 |
| AATTGAGTTA [SEQ ID NO. 2] | |
| AATTCGGTCAACAATTATAGTTGATTA | WGAN-GP_sfgfp_ |
| TCGCGTAAAAGTATGCTAC | round1_2 |
| CCTTAAGTTA [SEQ ID NO. 3] | |
| AATTCGAGAACATGTTTGCGTTGAAAA | WGAN-GP_sfgfp_ |
| ACTTGTAAGGTTGGGCTAA | round1_29 |
| AATAGCGTAA [SEQ ID NO. 4] | |

Table 1 provides a list of synthetic prokaryotic promoters that are highly optimized for efficient use in E. coli (on a plasmid or on the bacterial chromosome) to encode antibodies, antibody derivatives, or proteins/polypeptides.

TABLE 2

| Synthetic Terminator Sequence (5′-3′) | Name |
|---|---|
| AAAAAAAAGGTATTGTCAGTGCGAAAGCACTGACAAT | T80 |
| ACCTTTTTTTT [SEQ ID NO. 5] | |
| AAAAAAAAGGTTGTTTCAGTGCTTTCGCACTGAAACA | T11 |
| ACCTTTTTTTT [SEQ ID NO. 6] | |
| CTCGGTACCAAATTCCAGAAAAGAGACGCTTTCGAGC | L3S2P11 |
| GTCTTTTTTCGTTTTGGTCC [SEQ ID NO. 7] | |
| CTCGGTACCAAAGACGAACAATAAGACGCTGAAAAGC | L3S2P55 |
| GTCTTTTTTCGTTTTGGTCC [SEQ ID NO. 8] | |

Table 2 provides a list of synthetic prokaryotic terminators that are highly optimized for efficient use in E. coli (on a plasmid or on the bacterial chromosome) to encode antibodies, antibody derivatives, or proteins/polypeptides.

TABLE 3
E. coli-optimized, anti-survivin nanobody

```
5′ atgggtgaagttcagctggttgaatctggtggtggttctgttcaggcgggtggttctctg
3′ tacccacttcaagtcgaccaacttagaccaccaccaagacaagtccgcccaccaagagac
        M G E V Q L V E S G G G S V Q A G G S L
      cgtctgtcttgcgcggcgtctggtgtttctaacaccgacatgatcatgatctggttccgt
      gcagacagaacgcgccgcagaccacaaagattgtggctgtactagtactagaccaaggca
        R L S C A A S G V S N T D M I M I W F R
      caggcgccgggtaaagaacgtgaaggtgttctggcggcgatctacaaaaactctacctac
      gtccgcggcccatttcttgcacttccacaagaccgccgctagatgtttttgagatggatg
        Q A P G K E R E G V L A A I Y K N S T Y
      tacgcggactctgttaaaggtcgtttcaccatctctcaggacaacgcgaaaaacaccgtt
      atgcgcctgagacaatttccagcaaagtggtagagagtcctgttgcgctttttgtggcaa
        Y A D S V K G R F T I S Q D N A K N T V
      tacctgcagatgaactctctgaaaccggaagacaccgcgatctactactgcgcggcgatc
      atggacgtctacttgagagactttggccttctgtggcgctagatgatgacgcgccgctag
        Y L Q M N S L K P E D T A I Y Y C A A I
      cgtgcggttatcggtcgtcacatccgtccgcacatctactggggtcagggtacccaggtt
      gcacgccaatagccagcagtgtaggcaggcgtgtagatgaccccagtcccatgggtccaa
        R A V I G R H I R P H I Y G Q G T Q V
      accgttggtggtggttctcaccaccaccaccaccacggtggttcttaatga 3′ [SEQ ID NO 9]
      tggcaaccaccaccaagagtggtggtggtggtggtgccaccaagaattact 5′ [SEQ ID NO 10]
        T V G G G S H H H H H H G G S - - [SEQ ID NO 11]
```

What is claimed is:

1. A system for generating and delivering proteins to eukaryotic cells comprising a bacterium that has been engineered to be invasive and has been engineered to have a least one expression cassette encoding a protein that is exogenous to the bacterium, wherein the transcription of the nucleic acid encoding the exogenous protein is under the control of a prokaryotic promoter and terminator.

2. The system for generating and delivering proteins to eukaryotic cells according to claim 1 wherein the prokaryotic promoter and terminator are synthetic, whereby the promoter or terminator have transcription-promoting or transcription-terminating activity in E. coli.

3. The system for generating and delivering proteins to eukaryotic cells according to claim 1 wherein the encoded protein is an antibody or an antibody derivative.

4. The system for generating and delivering proteins to eukaryotic cells according to claim 3, wherein the antibody or antibody derivative consists essentially of a single protein domain extracted from a multi-domain antibody.

5. (canceled)

6. The system for generating and delivering proteins to eukaryotic cells according to claim 3, wherein the antibody or antibody derivative comprises a biologically active peptide that has an effect on a living organism, tissue, cell, or biochemical process, grafted onto an Fc domain or other antibody domain (e.g., a peptibody) of the antibody or antibody derivative.

7. (canceled)

8. The system for generating and delivering proteins to eukaryotic cells according to claim 3 wherein a structural domain of the antibody or antibody derivative has an amino acid sequence that binds an epitope to target an intracellular protein and wherein the intracellular protein is a therapeutically relevant protein or therapeutically relevant as a binding target for the antibody or antibody derivative.

9. The system for generating and delivering proteins to eukaryotic cells according to claim 3 wherein the antibody or antibody derivative forms a complex with a target protein, thereby modulating a specific activity or cellular pathway in a eukaryotic cell by rendering the target protein biologically inactive due to the antibody obscuring, occupying, or otherwise interfering with a binding site or epitope important for target protein interactions with other molecules, and wherein the uncomplexed target is biologically active.

10. (canceled)

11. The system for generating and delivering proteins to eukaryotic cells according to claim 1 wherein the intracellular factor that is bound by the antibody or antibody derivative inside a cancer cell is a mutated HRAS, NRAS, or KRAS protein.

12. (canceled)

13. The system for generating and delivering proteins to eukaryotic cells according to claim 3 wherein binding by the antibody or antibody derivative modulates a specific activity or cellular pathway thereby enhancing the therapeutic efficacy of a chemotherapeutic agent or other therapy administered to or performed on a subject.

14. The system for generating and delivering proteins to eukaryotic cells according to claim 3 wherein the antibody or antibody derivative contains a region that binds an epitope on an apoptosis-regulating or apoptosis-related protein.

15. The system for generating and delivering proteins to eukaryotic cells according to claim 14 wherein the antibody or antibody derivative contains a region that binds survivin (BIRC5), BCL-2, MCL-1, XIAP, BRUCE, or any other inhibitor of apoptosis (IAP)-family protein or protein that contains one or more characteristic BIR domains.

16. The system for generating and delivering proteins to eukaryotic cells according to claim 3 wherein the antibody or antibody derivative contains a region that binds an epitope on a viral, bacterial, protozoan, or fungal protein, whereby binding the epitope inhibits viral, bacterial, protozoan, or fungal replication.

17. The system for generating and delivering proteins to eukaryotic cells according to claim 1 wherein the bacterium is a nonpathogenic bacterium engineered to have at least one invasion factor to facilitate entry of the nonpathogenic bacterium into a eukaryotic cell or cause release of the nonpathogenic bacterium from a eukaryotic cell phagosome.

18. The system for generating and delivering proteins to eukaryotic cells according to claim 17 wherein the invasion factor is encoded by an inv, hlyA, or hlyE gene or any fragment or chimeric or recombinant version thereof.

19. The system for generating and delivering proteins to eukaryotic cells according to claim 17 wherein the invasion factor is a chimeric, recombinant invasion protein comprising the non-binding domains of an invasin protein fused to a binding domain from a heterologous protein.

20. The system for generating and delivering proteins to eukaryotic cells according to claim 17 wherein the binding domain from a heterologous protein is selected from a binding domain of GalNAc binding proteins, lectins, the group of cell adhesion molecules (CAMs), the group of sulfated glycosaminoglycan (GAG)-binding proteins, selectins, integrins, laminin, cadherins, fibronectin, collagens, thrombospondin, vitronectin, tenascin, apolipoproteins B, E, and A-V, lipoprotein lipase, hepatic lipase, Siglecs, galectins, immunoglobulins, and annexins, FimH, papG, PrsG, Afa-IE, DraA, MrpH, RodA, Mp1, hydrophobins, heat shock protein, CspA, hemagglutinin, neuraminidase, capsid protein, glycoproteins, and envelope proteins.

21. (canceled)

22. The system for generating and delivering proteins to eukaryotic cells according to claim 1 wherein the bacterium is a nonpathogenic bacterium engineered to have at least one cellular targeting factor (surface-expressed moiety).

23. (canceled)

24. (canceled)

25. The system for generating and delivering proteins to eukaryotic cells according to claim 1, wherein the expression cassette is on a plasmid having a length of approximately 7,000 base pairs or less, whereby a reduction in plasmid size relative to a larger plasmid decreases plasmid-induced burden to the host bacterial cells, thereby increasing the bacterial growth rate.

26. (canceled)

27. The system for generating and delivering proteins to eukaryotic cells according to claim 1 wherein the protein encoded by the invasive bacterium is delivered to the cytoplasm of a target eukaryotic cell and wherein the protein is functional in a eukaryotic cell and increases the level of protein in the target eukaryotic cell to supplement a clinically significant deficiency in the endogenous level of said protein.

28. (canceled)

29. (canceled)

30. (canceled)

31. (canceled)

32. (canceled)

33. A method for replacing or supplementing an endogenous eukaryotic protein in a eukaryotic target cell comprising the step of contacting the eukaryotic target cell with a bacterium comprising an expression cassette encoding and producing a eukaryotic protein in need of replacement or supplementation in the target cell wherein transcription of the protein is under the control of a prokaryotic promoter and wherein the bacterium is a nonpathogenic bacterium that is engineered to be invasive to the eukaryotic cell and wherein the exogenously delivered bacterially expressed eukaryotic protein has the same biological or biochemical activity as the endogenous eukaryotic protein and that this activity is present in the eukaryotic cell (i.e., the protein carries out its normal function).

34. The system for generating and delivering proteins to eukaryotic cells according to claim 17 wherein the invasion factor that facilitates entry of the nonpathogenic bacterium into a eukaryotic cell is encoded by the entry protein FimH, OmpA, IbeA, IbeB, IbeC, Opc, PilA, PilB, LOS, Lmb, FbsA, IagA, Vsp1, OspA, 70-kDa PBP, enolase, Isc1, Yps3p, Stx, type 3 secretion system-injected factors, EspF, Map, EspG), or any fragment or chimeric or recombinant version thereof.
